(19) United States
(12) Patent Application Publication
(10) Pub. No.: US 2021/0255110 A1
(43) Pub. Date: Aug. 19, 2021

(54) METHOD FOR TESTING BODY FLUIDS

(71) Applicant: MILKSTRIP LTD., Rehovot (IL)

(72) Inventors: Avital BECK, Rehovot (IL); Hadas SHATZ-AZOULAY, Yavne (IL)

(21) Appl. No.: 17/192,193

(22) Filed: Mar. 4, 2021

Related U.S. Application Data
(63) Continuation-in-part of application No. PCT/IL2019/050988, filed on Sep. 4, 2019.
(60) Provisional application No. 62/726,527, filed on Sep. 4, 2018.
(60) Provisional application No. 62/831,806, filed on Apr. 10, 2019.

(51) Int. Cl. G01N 21/78; C12Q 1/28; G01N 33/487; G01N 21/80

(52) U.S. Cl. CPC G01N 21/78; C12Q 1/28; G01N 2021/7759; G01N 21/80; G01N 33/487

(57) ABSTRACT

The present invention provides devices, test strip and methods for testing breastmilk and providing feedback for improving the quality thereof.

### FIELD OF THE INVENTION

The present invention generally relates to testing body fluids. More particularly, the present invention relates to testing breastmilk and providing feedback for improving the quality of the breastmilk.

### BACKGROUND

It is presently held that breastmilk is nutritionally superior to any man-made formula, and that breast-feeding generally appears quite beneficial for both the mother and the infant. Unlike infant formula, which is standardized within a very narrow range of composition, human breastmilk composition varies within each feeding, over lactation, environmental factors and between mothers and populations.

However, since the nutritional quality of human breastmilk depends on the maternal diet for vitamins, fatty acid composition and more, breast-feeding presents concerns for the mother, in that the maternal diet effects can be passed through to the infant.

Several scientific papers address the issue of stored (frozen or refrigerated) breastmilk. The consensus is that although breastmilk is usually suitable for consumption after storage (according to storage guidelines), there are some cases in which the breastmilk is considered unsafe or unsuitable for consumption. Most of these cases involve an elevation in aero-bacterial load. The numbers vary from 10⁴-10⁶ CFU/ml depending on the research and the type of bacteria found.

Another essential factor in breastmilk is sugar. Many sugar types/forms are found in breastmilk, the main is lactose (98%). Notably, fresh breastmilk does not contain galactose. However, various data support the notion that galactose is accumulated in breastmilk with bacterial growth. Hence, determination of galactose concentrations is indicative of bacterial overload. For instance, C. Schwab M. Ganzle (FEMS Microbiology Letters, 2011, Vol. 315(2): 141-148) shows that lactose and glucose were completely or partially utilized by all bacterial strains. Accumulation of galactose from lactose breakdown was detected in culture supernatants from L. acidophilus (approximately 3.5 mM) and L. mesenteroides, (lactic acid bacteria fermentation of human breastmilk oligosaccharide components, human breastmilk oligosaccharides and galactooligosaccharides. V. S. O'leary et al., (Applied AND Environmental Microbiology, 1976, pp. 89-94) shows that fermentation of lactose in breastmilk was accompanied by the release of free galactose.

Elevation in D and L-lactic acid concentration: S. Bhisikar et al. (Indian J Pediatr, 2018, 85(4): 272-275) exemplified the use of dornic acid test to detect breastmilk quality in breastmilk banks. They examined a significant bacterial growth in 21% of breastmilk samples with coagulase negative staphylococci and gram-negative organisms being the major contaminants seen prior to pasteurization. They demonstrated that 8° D (Dornic degrees) had 92% specificity and acceptable sensitivity of 72% in comparison to gold standard microbiological criteria, making it a good test for analyzing the quality of breastmilk before pasteurization.

S. Va'zquez-Roma'n et al. (Breastfeeding Medicine, Vol. 8(1), 2013) have classified breastmilk into three quality categories: top quality (acidity <4° D), intermediate (acidity between 4° D and 7° D), and breastmilk unsuitable to be consumed (acidity >7° D). They suggest that measurement of dornic acidity could be considered a simple and economical method to select breastmilk to pasteurize in a human breastmilk bank based on quality and safety criteria. However, dornic acidity is currently measured using titration assays which is inapplicable for test strips.

It has been shown that the features of lactose intolerance highly resemble symptoms of infantile colic. When the levels of lactose in the lumen of the small intestine exceed the capacity of the available lactase: the gastrointestinal (GIT) microbial flora ferment excess lactose, particularly in the colon, producing gases (carbon dioxide, hydrogen and methane) and acid; lactose fermentation products in the colon increase the osmolarity of the lumen's contents, therefore increasing the volume of water that must be retained in the lumen; any unsplit lactose still remaining in the colon also mandates retention of water by the colon; GIT transit time is shortened; depending on severity, mucus may be evident in stools due to irritation of the GIT mucosa.

Hence the presenting symptoms of lactose overload in breastfed babies are: excessive flatus (a “windy” baby); frequent explosive watery stools which may be yellow or green, depending on severity; “colic” (defined in this case as crying due to overproduction of intestinal gases); unsettled baby; perianal acid burns may occur; and stool may be mucousy.

There are many reasons for lactose overload in breastfed babies. For instance, Woolridge M W et al. (Lancet, 1988, 2:382) and Noble R. et al. (http://www.health-e-learning.com/resources/articles, January 1998) describe a type of lactose overload that is due entirely to low fat feeds. This may arise if babies are not permitted to nurse long enough during feeds, such as when feeds are clock-regulated, or if mothers need to shorten feeds because they are painful. Babies may also be inefficient feeders who are unable to milk the breast well enough to extract the fattier breastmilk that comprises the end of feeds (hind breastmilk). Low-fat feeds cause fast gastric clearance, hence overloading the small intestine's capacity to metabolize lactose.

Assessment of the mother's diet is another relevant consideration. Some mothers have minimal fat/high sugar intakes, others simply eat very little and very infrequently because they are stressed or “too busy”. Breakfast and lunch are commonly inadequate meals. There are also mothers who severely restrict their fat intakes to quickly lose weight gained in pregnancy. All these situations are undesirable for the mothers as much as for their breastfed babies. As such, inadequate fat intake in the maternal diet appears to make a direct contribution to the intensity of lactose overload symptoms, resulting in lower fat and higher lactose levels in their breastmilk. For instance, Lacovou et al. (Aliment Pharmacol Ther. 2018, Vol. 48(10): 1061-1073) describes that reduction in the intake of dietary FODMAPS (Fermentable Oligo-, Di-, Mono-Saccharides And Polyols) and elevation of fat in the diet of breastfeeding mothers is associated with greater improvement of the symptoms of infantile colic.

It has been shown that many substances found in breastmilk are associated with sleep habits such as iron, magnesium, caffeine and nicotine. For instance, Chollet D. et al. (Behav Genet., 2001, Vol. 31(5):413-25) showed that Mg²⁺ supplementation improves indicators of low magnesium status and inflammatory stress in adults with poor quality sleep. Depoortere H. et al. (J. Neuropsychobiology, 1993; 27(4):237-45) showed that a Mg²⁺-deficient diet resulted first in a significant increase of wakefulness at the expense of slow wave sleep, and eventually sleep was disorganized. Reintroducing Mg²⁺ in food and water, led to restoration of sleep organization and ECoG. Moreover, Forrest H. Nielsen (Nutrition Reviews, 2010, Vol. 68(6): 333-340) showed that magnesium deficiency is common among the general population, and is estimated to be found in about 60% of the western population, including in breastfeeding mothers.

Other articles address the issue of iron as a sleep support element. For instance, Dean at el. (Sleep Medicine, 2006, Vol. 7(8): 634-640) showed that dietary iron deficiency affected an increase in wakefulness during a specific circadian time point that would disturb sleep onset and progression. Moreover, iron-deficiency anemia (IDA) continues to be the most common single nutrient deficiency in the world. Peirano P. D. et al. (Sleep Medicine, 2010, Vol. 11(7): 637-642) showed that 20-25% of the world's infants have IDA, with at least as many having iron deficiency without anemia. Altered sleep patterns may represent an underlying mechanism that interferes with optimal brain functioning during sleep and wakefulness in former IDA children. In support thereof, Chantry C. J. et al. (Breastfeeding Medicine, 2007, Vol. 2(2): 63-73) showed that young children in the US fully breastfed for 6 months may be at increased risk of iron deficiency (ID) and anemia.

Nicotine in breastmilk has also been shown to lead to sleeping disorders in infants: Nicotine secreted into breastmilk has a potential to cause severe adverse effects on the newborn, which depends on the number of cigarettes consumed by the mother per day and also on the time interval between the last inhaled cigarette and the beginning of breastfeeding (since the half-life of nicotine in breastmilk is about two hours). For instance, Primo C. C. et al. (Rev. Paul. Pediatr., 2013, Vol. 31(3): 392-7) showed that the effects of maternal nicotine on infants are multiple, such as changes in sleep and wake patterns; reduction of iodine supply to the infant through breastmilk, leading to an increased risk of deficiency of iodine. Breastfed infants with a smoking or snuff taking mother are exposed to nicotine in breastmilk. Mennella J. A. et al. (Pediatrics, 2007, Vol. 120(3): 497-502) showed that both passive smoking at home and snuff-taking were associated with measurable nicotine levels in breastmilk, and breastfed infants spent significantly less time sleeping during the hours immediately after their mothers smoked.

Notably, commercial test strips are not suitable for measuring the various components and parameters within breastmilk for several reasons, the main is the presence of triglycerides in the breastmilk. Another reason is the relatively low or high amount of such components that need to be measured. For instance, standard protein test strips cannot be used to measure protein concentration within breastmilk since they react with triglyceride in the breastmilk, and since they are suitable for measuring protein ranges that are out of range of proteins in breastmilk.

Another problem of using commercial test strips for measuring breastmilk is color identification in the testing area(s)—breastmilk is not transparent and it has a strong and distinct color, which makes it difficult to notice color changes in the test strip.

Another issue with breastmilk and its testing, is that it is a complex media, in which some of the components are not free, but are rather attached to other components, such as B12 that is connected to cobalt. This makes it difficult to detect and analyze some of the components. Also, breastmilk contains numerous cells, such as immune cells, microbiome creating bacteria, stem cells and many more, as well as other factors such as enzymes and antibodies, all of which continue “working” after the breastmilk exits the breast and thus modify the content of the breastmilk during storage.

As such, a need exists for reliable and simple home-use analysis tools to evaluate breastmilk's conditions and quality. The present invention provides such strips that are compatible with breastmilk, overcome the inhibitory, color and blocking effects thereof, and are suitable for testing nutrients concentrations (either very high or very low) in breastmilk.

### SUMMARY

Breastmilk is the main, and many times the only, food source of infants, and is essential for growth and health thereof. As such, in order to provide infants with the best possible nutrition, it is advisable that the contents of breastmilk be monitored so as to enable determining whether the breastmilk is adequate or not, and/or if supplements are needed.

Thus, in accordance with some embodiments of the present invention, there is provided a test strip for testing a breastmilk sample, the test strip comprising a working surface that comprises at least one testing region with at least one testing medium, each one of said at least one testing medium is designed for measuring one component or parameter of the breastmilk, said at least one testing medium comprises: (i) at least one testing pad for enabling a single-step measuring procedure; and/or (ii) at least two testing pads for enabling a multi-step measuring procedure, wherein said testing regions are designed to undergo color change and/or texture/granulation variations as a result of a colorimetric reaction inducible when exposed to said breastmilk.

Furthermore, in accordance with some embodiments of the present invention, color change means a change in color intensity, hue, tone, shade, or any combination thereof.

Furthermore, in accordance with some embodiments of the present invention, the test strip comprises at least one of: breastmilk expiration testing region; colic symptom reduction testing region; vitamin C concentration testing region; macronutrients concentration testing region; baby sleep support testing region; contamination detection testing region; improvement in learning and memory testing region; and antioxidant detection testing region, or any combination thereof.

Furthermore, in accordance with some embodiments of the present invention, the test strip comprises at least two of: breastmilk expiration testing region; colic symptom reduction testing region; vitamin C concentration testing region; macronutrients concentration testing region; baby sleep support testing region; contamination detection testing region; improvement in learning and memory testing region; and antioxidant detection testing region, or any combination thereof.

Furthermore, in accordance with some embodiments of the present invention, the test strip comprises a breastmilk expiration testing region, and a colic symptom reduction testing region.

Furthermore, in accordance with some embodiments of the present invention, the test strip comprises a breastmilk expiration testing region, and a vitamin C concentration testing region.

Furthermore, in accordance with some embodiments of the present invention, the test strip comprises a breastmilk expiration testing region, and a macronutrients concentration testing region.

Furthermore, in accordance with some embodiments of the present invention, the test strip comprises a breastmilk expiration testing region, and a baby sleep support testing region.

Furthermore, in accordance with some embodiments of the present invention, the test strip comprises a breastmilk expiration testing region, and an improvement in learning and memory testing region.

Furthermore, in accordance with some embodiments of the present invention, the test strip comprises a breastmilk expiration testing region, and an antioxidant detection testing region.

Furthermore, in accordance with some embodiments of the present invention, the test strip comprises a breastmilk expiration testing region; a colic symptom reduction testing region; and a vitamin C concentration testing region.

Furthermore, in accordance with some embodiments of the present invention, the test strip comprises a breastmilk expiration testing region; a colic symptom reduction testing region; and a macronutrients concentration testing region.

Furthermore, in accordance with some embodiments of the present invention, the test strip comprises a breastmilk expiration testing region; a colic symptom reduction testing region; and a baby sleep support testing region.

Furthermore, in accordance with some embodiments of the present invention, the test strip comprises a breastmilk expiration testing region; a colic symptom reduction testing region; and improvement in learning and memory testing region.

Furthermore, in accordance with some embodiments of the present invention, the test strip comprises a breastmilk expiration testing region; a colic symptom reduction testing region; and an antioxidant detection testing region.

Furthermore, in accordance with some embodiments of the present invention, the test strip comprises: a breastmilk expiration testing region; a colic symptom reduction testing region; a vitamin C concentration testing region; a macronutrients concentration testing region; a baby sleep support testing region; a contamination detection testing region; an improvement in learning and memory testing region; and an antioxidant detection testing region.

Furthermore, in accordance with some embodiments of the present invention, the breastmilk expiration testing region comprises a pH test pad, and a bacterial load marker pad.

Furthermore, in accordance with some embodiments of the present invention, the bacterial load marker pad is selected from a galactose test pad, a lactic acid test pad, and a protein test pad, or any combination thereof.

Furthermore, in accordance with some embodiments of the present invention, the colic symptom reduction testing region comprises a triglyceride test pad and optionally a lactose test pad.

Furthermore, in accordance with some embodiments of the present invention, the vitamin C concentration testing region comprises one vitamin C test pad or two different-color vitamin C test pads.

Furthermore, in accordance with some embodiments of the present invention, the macronutrients concentration testing region comprises a lactose test pad, a triglycerides test pad, and a protein test pad.

Furthermore, in accordance with some embodiments of the present invention, the baby sleep support testing region comprises at least one of: an iron pad, a magnesium pad, a caffeine pad and a nicotine pad.

Furthermore, in accordance with some embodiments of the present invention, the contamination detection testing region comprises at least one of: a caffeine pad, a nicotine pad and an alcohol pad.

Furthermore, in accordance with some embodiments of the present invention, the improvement in learning and memory testing region comprises at least one of: an iron pad, a magnesium pad and a sialic acid pad.

Furthermore, in accordance with some embodiments of the present invention, the antioxidant detection testing region comprises a vitamin C pad and at least one of: malondialdehyde (MDA) pad and glutathione peroxidase pad.

Furthermore, in accordance with some embodiments of the present invention, the working surface comprises: (a) a sample receiving reservoir; (b) testing regions comprising at least one testing medium, each one of said at least one testing medium comprises: (i) at least one testing pad for enabling a single-step measuring procedure; and/or (ii) at least two testing pads for enabling a multi-step measuring procedure; and (c) fluid continuous transporting routs; wherein said fluid continuous transporting routs connect said sample receiving reservoir to each one of said testing regions and are designed for directing breastmilk from said breastmilk sample placed within said reservoir to said testing regions.

Furthermore, in accordance with some embodiments of the present invention, a system for testing breastmilk to determine whether it: (i) is spoiled or not, and/or (ii) has deficiencies in certain nutrients, and for producing recommendations for nutritional components, quantities and consumption frequency for overcoming said deficiencies and improving breastmilk's quality, the system comprises: (a) the test strip described above; (b) optionally, a color chart/a reference color card; (c) a user device comprising a scanning element for scanning said testing regions of said test strip; and (d) a cloud server including a breastmilk qualification program, said cloud server designed to: (i) receive user's data and scan(s) of said testing regions via said user device; (ii) apply machine learning algorithms to incorporate said data and scan(s); and (iii) provide the following outputs: whether the breastmilk is spoiled or not, and/or possible nutrients deficiencies, and recommendations for nutritional components, quantities and consumption frequency for overcoming said nutrients deficiencies for improving breastmilk's quality, wherein said scan(s) showing color change intensities induced by colorimetric reaction(s) in said testing medium of said test strip, and wherein said machine learning algorithms monitor the color change in said testing regions, and based thereon provide said output.

Furthermore, in accordance with some embodiments of the present invention, the user device is an electronic communication device selected from a computer, cellular phone, or any other device that can access, provide, transmit, receive, and modify information over wired or wireless networks.

Furthermore, in accordance with some embodiments of the present invention, the user's data comprises at least one of: weight of the mother, weight of the newborn, age of the mother, type of birth, number of newborns, ethnic origin, diet food preferences and/or avoidances, whether the mother is a vegan or vegetarian, blood sugar level, the mother's temperature, and the day of week and month of the year on which the breastfeeding mother enters the data, or any combination thereof.

Furthermore, in accordance with some embodiments of the present invention, a Breastmilk Qualification Model I is trained to provide an output stating whether the breastmilk is spoiled or not and/or whether there any nutrients' deficiencies.

Furthermore, in accordance with some embodiments of the present invention, a Breastmilk Qualification Model II is trained to provide output recommendations for nutritional components, quantities and consumption frequency for overcoming said nutrients deficiencies and subsequently for improving breastmilk's quality.

Furthermore, in accordance with some embodiments of the present invention, there is also provided a method for testing breastmilk to determine whether it: (i) is spoiled or not, and/or (ii) has deficiencies in certain nutrients, and for producing recommendations for nutritional components, quantities and consumption frequency for overcoming said deficiencies and improving breastmilk's quality, the method comprises: (1) providing the test strip described above; (2) optionally, providing a color chart/a reference color card; (3) providing a user device comprising a scanning element for scanning said testing regions of said test strip; (4) testing said breastmilk using said at least one test strips and scanning said testing regions using said user device; (5) applying machine learning algorithms at an online remote cloud server, said algorithms are designed to incorporate uploaded user's data and data obtained from said user device and provide an output stating whether the breastmilk is spoiled or not and/or has deficiencies in certain nutrients, and further provide an output with recommendations for nutritional regimen to improve breastmilk's quality; and (6) providing an online interface to access said outputs.

Furthermore, in accordance with some embodiments of the present invention, the test strip is designed to determine at least one of the following: breastmilk expiration, colic symptom reduction, vitamin C concentration, macronutrients concentration, baby sleep support, contamination detection, improvement in learning and memory, and antioxidant detection, or any combination thereof.

Furthermore, in accordance with some embodiments of the present invention, the test strip for determining breastmilk expiration comprises a pad for measuring galactose concentration and a pad for measuring pH.

Furthermore, in accordance with some embodiments of the present invention, the test strip for determining colic symptom reduction comprises a pad for measuring triglyceride concentration and optionally also a pad for measuring lactose concentration.

Furthermore, in accordance with some embodiments of the present invention, the test strip for determining vitamin C concentration comprises one pad for measuring vitamin C concentration or two different-color vitamin C test pads.

Furthermore, in accordance with some embodiments of the present invention, the test strip for determining macronutrients concentration comprises a pad for measuring the concentration of lactose, a pad for measuring the concentration of triglyceride and a pad for measuring the concentration of protein.

Furthermore, in accordance with some embodiments of the present invention, the test strip for determining baby sleep support comprises pads for measuring the concentration of iron, magnesium, nicotine and caffeine.

Furthermore, in accordance with some embodiments of the present invention, the test strip 100 for determining contamination detection comprises pads for measuring the concentration of alcohol, nicotine and caffeine.

Furthermore, in accordance with some embodiments of the present invention, the test strip 100 for determining improvement in learning and memory comprises pads for measuring the concentration of iron, magnesium and sialic acid.

Furthermore, in accordance with some embodiments of the present invention, the test strip 100 for determining antioxidant detection comprises a pad for measuring the concentration of vitamin C and pad(s) for measuring either malondialdehyde (MDA) and/or glutathione peroxidase.

Furthermore, in accordance with some embodiments of the present invention, the method further comprising the following steps: accumulating and monitoring data from at least one user device; and propagating said data to an online remote server.

### BRIEF DESCRIPTION OF THE FIGURES

FIG. 1 illustrates a test strip for measuring pH and concentrations of various components in a body fluid sample (e.g. breastmilk) in accordance with some embodiments of the present invention.

FIG. 2 presents a schematic block diagram of an algorithm in accordance with some embodiments of the present invention for determination of breastmilk usability based on pH and galactose or Dornic degrees.

FIG. 3 presents a schematic block diagram of a breastmilk qualification system in accordance with some embodiments of the present invention.

FIG. 4 is a block diagram depicting a high-level overview of one possible embodiment of a system according to the present invention.

FIG. 5 is a flow diagram depicting the functionality of a data acquisition module according to some embodiments of the present invention.

FIG. 6 is a flow diagram depicting the functionality of a training module according to some embodiments of the present invention;

FIG. 7 is a flow diagram depicting the function of Model I after the training stage in accordance with some embodiments of the present invention;

FIG. 8 is a flow diagram, depicting the function of Model II after the training stage in accordance with some embodiments of the present invention; and

FIG. 9 is a flow diagram depicting the functionality of a decision module according to some embodiments of the present invention.

### DETAILED DESCRIPTION

Since breastmilk is the main or only nutrition source of infants, it is vital to test that the quality of breastmilk given to an infant and to verify that it is: not foul, does not contain undesired and harmful ingredients, and moreover that it contains all required and essential ingredients for proper growth and health. Preferably, such testing is carried out prior to the administration of breastmilk that was pumped and stored to an infant. Another preference is to test the breastmilk during live breastfeeding to validate the breastmilk quality during breastfeeding.

Thus, in one aspect, the present invention provides test strip apparatus 100 to check the ratios of various components in breastmilk, such as lactose and fat (triglycerides), by any suitable technique, such as enzymatic reactions. For instance, since presence of certain amounts of bacteria is indicative of spoiled breastmilk, measuring galactose concentration and pH may provide insights regarding whether the breastmilk if spoiled or not.

Another example is for infantile colic: since normal average lactose concentration is about 7 mg/dl and fat content is about 3.5 mg/dl, a ratio of lactose/fat of over 2 is an indication of an increased risk for lactose overload and for infantile colic. Accordingly, mothers with high lactose/fat ratios in their breastmilk would thus be advised to find supplement breastmilk or other supplements, and would further be advised to amend their diets to fattier and less sugar-rich diets in order to improve their breastmilk quality.

Accordingly, in a first aspect, the present invention provides a test strip 100 for testing a breastmilk sample, the test strip 100 comprising a working surface 104 that comprises at least one testing region 112 with at least one testing medium, each one of said at least one testing medium is designed for measuring one component or parameter of the breastmilk, said at least one testing medium comprises: (i) at least one testing pad for enabling a single-step measuring procedure; and/or (ii) at least two testing pads for enabling a multi-step measuring procedure, wherein said testing regions 112 are designed to undergo color change and/or variations in the texture/granulation as a result of a colorimetric reaction inducible when exposed to said breastmilk.

The term “color change” as used herein includes any change in the color, such as intensity, hue, tone, shade, etc., and any combination thereof.

In certain embodiments, the test strip 100 of the invention comprises an extension 106, e.g. arm, designed to be held by a user when using the strip.

In a specific embodiment, the test strip 100 of the invention has a unique configuration, in which case the test strip 100 comprises a working surface 104 that comprises: (i) a sample receiving reservoir 108; (ii) testing regions 112 comprising at least one testing medium, each one of said at least one testing medium comprises: (i) at least one testing pad for enabling a single-step measuring procedure; and/or (ii) at least two testing pads for enabling a multi-step measuring procedure; and (iii) fluid continuous transporting routs 110; wherein said fluid continuous transporting routs 110 connect said sample receiving reservoir 108 to each one of said testing regions 112 and are designed for directing breastmilk from said breastmilk sample placed within said reservoir 108 to said testing regions 112.

In certain embodiments, said transporting routs 110 are grooves, channels—open or closed, fibers, conductive material, or any other material or structure that can week and transport liquid from the reservoir 108 to said testing regions 112. The dimensions, i.e., the diameter, of the transporting routs 110 may vary depending on the desired flow rate.

FIG. 1 illustrates a test strip 100 according to the above specific configuration for measuring various components and parameters, e.g., pH and the concentrations of various components in a breastmilk sample (fresh, pumped or frozen) in accordance with some embodiments of the present invention. Such parameters may be selected from the pH; concentrations of fat, lactose, galactose and protein; the presence of vitamin C; and concentrations of nutrients including lactose, triglycerides, protein, lactic acid and Dornic acidity. Thus, in accordance with some embodiments of the present invention, the above components and correlations between selected components aid in determining whether a breastmilk sample is, e.g., spoiled or not.

In certain embodiments, when the test strip 100 comprises a sample receiving reservoir 108, breastmilk can be dripped thereon, and is then transported via the transporting routs 110 to the testing regions 112, where it reacts with one or more reagents in designated pads. Alternatively, the test strip 100 may be dipped into a breastmilk sample, or the breastmilk may be dripped directly onto the testing regions 112, where reaction occurs. The reaction of the breastmilk with the reagent(s) in the pad(s) within the testing regions 112 results with a color differentiation that can be observed and compared to a color chart for analysis. This can be done manually or automatically using a computer-based program.

In certain embodiments, the test strip 100 provides instant feedback. Alternatively, or in addition, it may require some time for the reaction to complete.

In specific embodiments of the test strip 100 of the invention, the test strip 100 comprises various regions for testing different components. For instance, the test strip 100 may comprise any one of the following regions: breastmilk expiration testing region; colic symptom reduction testing region; vitamin C concentration testing region; macronutrients concentration testing region; baby sleep support testing region; contamination detection testing region; improvement in learning and memory testing region; and antioxidant detection testing region, or any combination thereof.

Non-limiting examples of possible combinations of test strips 100 according to the invention are: (1) a breastmilk expiration testing region, and a colic symptom reduction testing region; (2) a breastmilk expiration testing region, and a vitamin C concentration testing region; (3) a breastmilk expiration testing region, and a macronutrients concentration testing region; (4) a breastmilk expiration testing region, and a baby sleep support testing region; (5) a breastmilk expiration testing region, and an improvement in learning and memory testing region; (6) a breastmilk expiration testing region, and an antioxidant detection testing region; (7) a breastmilk expiration testing region; a colic symptom reduction testing region; and a vitamin C concentration testing region; (8) a breastmilk expiration testing region; a colic symptom reduction testing region; and a macronutrients concentration testing region; (9) a breastmilk expiration testing region; a colic symptom reduction testing region; and a baby sleep support testing region; (10) a breastmilk expiration testing region; a colic symptom reduction testing region; and improvement in learning and memory testing region; (11) a breastmilk expiration testing region; a colic symptom reduction testing region; and an antioxidant detection testing region; (12) a breastmilk expiration testing region; a colic symptom reduction testing region; a vitamin C concentration testing region; a macronutrients concentration testing region; a baby sleep support testing region; a contamination detection testing region; an improvement in learning and memory testing region; and an antioxidant detection testing region; (13) a pH test pad, and a bacterial load marker pad, wherein said bacterial load marker pad is optionally selected from a galactose test pad, a lactic acid test pad, and a protein test pad, or any combination thereof; (14) a triglyceride test pad and optionally a lactose test pad; (15) one vitamin C test pad or two different-color vitamin C test pad; (16) a lactose test pad, a triglycerides test pad, and a protein test pad; (17) at least one of: an iron pad, a magnesium pad, a caffeine pad, a choline pad and a nicotine pad; and (18) at least one of: a caffeine pad, a nicotine pad and an alcohol pad.

Each testing region comprises unique pads, each designed to measure one or more specific components of the breastmilk. These specific components are associated with a specific condition, and the combination of two or more of these specific components is indicative of the occurrence or absence of such specific condition. Non-limiting examples of such combinations of components are: pH and bacterial load for evaluating whether the breastmilk is spoiled or not; lactose and triglyceride for evaluating colic symptom reduction; and lactose, triglycerides and proteins for evaluating macronutrients concentration.

It should be noted that the test strip 100 of the invention is designed to identify the presence and/or measure the content of certain components and parameters within breastmilk. The number of components and parameters being examined/measured is determined according to the type of required analysis. For instance, a test strip may measure a single component or parameter, or may measure two, three, four, five, six or more components and parameters. The measurement may be carried out by using a single test strip 100. Alternatively, a user may use one, two, three or more different test strip 100, each measuring different (or same) components and parameters, which will be used to reach a final conclusion regarding the tested breastmilk.

Accordingly, in certain embodiments of the test strip 100 of the invention, breastmilk expiration testing region comprises a pH test pad, and a bacterial load marker pad, macronutrients concentration. In specific embodiments, the bacterial load marker pad is selected from a galactose test pad, a lactic acid test pad, and a protein test pad, or any combination thereof.

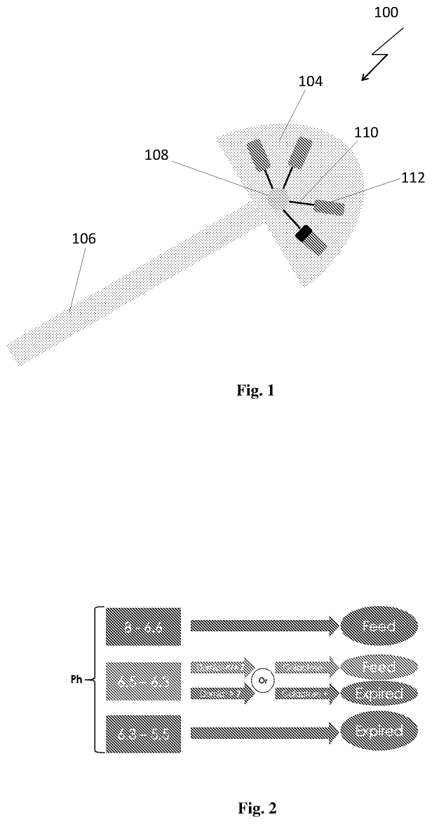

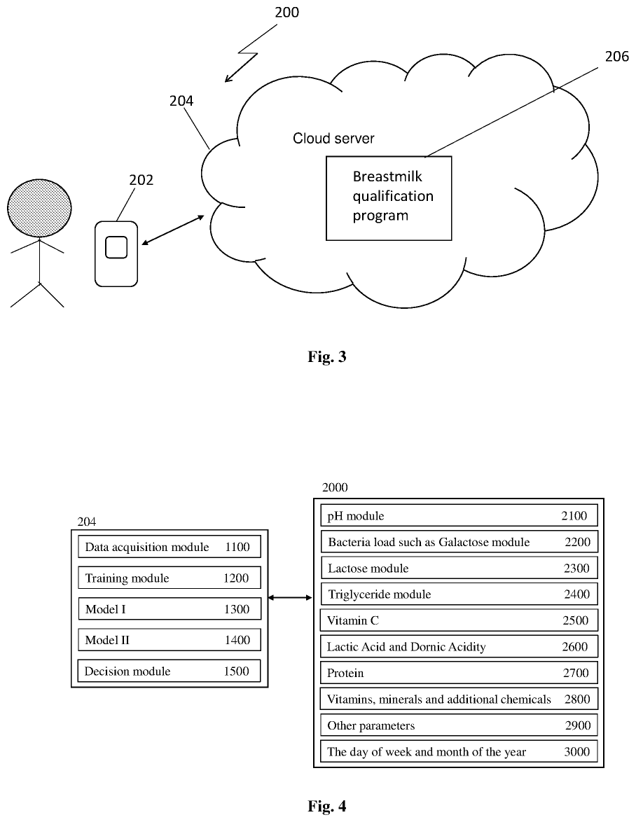

For example, FIG. 2 illustrates an algorithm according to the invention for breastmilk usability (spoiled or not) based on a combination of pH measurements and either galactose or Dornic degrees.

In other embodiments of the test strip 100 of the invention: a colic symptom reduction testing region comprises a lactose test pad and a triglyceride test pad; a vitamin C concentration testing region comprises a vitamin C test pad; macronutrients concentration testing region comprises a lactose test pad, a triglycerides test pad, and a protein test pad; baby sleep support testing region comprises at least one of: an iron pad, a magnesium pad, a caffeine pad and a nicotine pad; and/or contamination detection testing region comprises at least one of: a caffeine pad, a nicotine pad and an alcohol pad. For example, a contamination test region may comprise pads for detecting breastmilk contaminants such as alcohol, nicotine, caffeine and THC.

In yet other embodiments of the test strip 100 of the invention, improvement in learning and memory testing region comprises at least one of: an iron pad, a magnesium pad and a sialic acid pad. For example, an improvement in learning and memory testing region may comprise pads for measuring concentration of magnesium, sialic acid and iron.

In further embodiments of the test strip 100 of the invention, antioxidant detection testing region comprises a vitamin C pad and at least one of: malondialdehyde (MDA) pad and glutathione peroxidase pad. Notably, detection of antioxidants level in breastmilk is indicative of proper growth and development.

It should be noted that the test regions of the test strip 100 according to the invention may comprise any know examination pad, for any type of substance besides the ones mentioned and exemplified herein, such as for detection of free fatty acids, vitamin B12 and nitrogen.

It should be noted that the different testing regions of the test strip 100 according to the invention, for testing the different components within the breastmilk are based on any suitable method, such as, but not limited to, chemical methods or enzymatic methods. In specific exemplary embodiments, galactose concentration in the breastmilk is measured via chemical methods or enzymatic methods. In further specific exemplary embodiments, vitamin C concentration in the breastmilk is measured via chemical methods.

In certain embodiments, the reactions in each testing region are colorimetric reaction that leads to a color change within each region, which is indicative of the presence or absence of the tested component. In specific embodiments, the color changing is gradient, i.e. the stronger color change indicates stronger reaction.

In specific embodiments of the test strip 100 of any of the embodiments above, vitamin C concentration within the breastmilk is tested via food colors changing the reaction color depending on the vitamin C concentration.

As noted above, the test strip 100 of the invention can be used to measure and identify various components and parameters within breastmilk. In specific embodiments, such identifications are based on color change due to a reaction at testing regions 112 within the test strip 100. The identification of such color modifications can be carried out manually, or preferably by an electronic device that can capture a picture of the test strip 100, and subsequently a computer-based software or algorithm can analyze the picture and determine in which testing regions a reaction occurred and eventually determine the appropriate result(s).

Accordingly, the present invention provides a system 200 for testing breastmilk to determine whether it: (i) is spoiled or not, and/or (ii) has deficiencies in certain nutrients, and for producing recommendations for nutritional components, quantities and consumption frequency for overcoming said deficiencies and improving breastmilk's quality, the system 200 comprises: (1) the test strip 100 according to the invention; (2) a user device 202 comprising a scanning element for scanning said testing regions 112 of said test strip 100; (3) a cloud server 204 including a breastmilk qualification program 206, said cloud server 204 designed to: (i) receive user's data and scan(s) of said testing regions 112 via said user device 202; (ii) apply machine learning algorithms to incorporate said data and scan(s); and (iii) provide the following outputs: whether the breastmilk is spoiled or not, and/or possible nutrients deficiencies, and recommendations for nutritional components, quantities and consumption frequency for overcoming said nutrients deficiencies for improving breastmilk's quality, wherein said scan(s) showing color change intensities induced by colorimetric reaction(s) in said testing medium of said test strip, and wherein said machine learning algorithms monitor the color change in said testing regions 112, and based thereon provide said output. In specific embodiments, the system 200 further comprises a color chart/a reference color card that accompanies the test strip 100 in order to have a basis for comparison of the color obtained in the test strip 100. This color chart may be an independent card or stick, or may be an integral part of the test strip 100, e.g., located on its handle or at an area near the test region(s).

FIG. 3 presents a schematic block diagram of a breastmilk qualification system 200 in accordance with some embodiments of the present invention. Breastmilk qualification system 200 comprises at least one user device 202 and a cloud server 204 including a breastmilk qualification program 206. The at least one user device 202 may be an electronic communication device such as but not limited to a computer, cellular phone, and other devices that can access, provide, transmit, receive, and modify information over wired or wireless networks. The test strip 100 may be scanned via the at least one user device 202, and the breastmilk qualification program 206 in the cloud server 204 may process the data, i.e., the color intensity of the colorimetric reactions, and additional user's data, personal info (as described below) by means of machine learning algorithms to produce nutrition instructions.

Accordingly, in specific embodiments of the system 200 of the invention, the user device 202 is an electronic communication device selected from a computer, cellular phone, or any other device that can access, provide, transmit, receive, and modify information over wired or wireless networks. In specific embodiments, the user device 202 comprises a camera or scanner capable of acquiring an image of the test strip 100 or of its testing regions 112.

In certain embodiments of the system 200 according to the invention, the user's data comprises at least one of: weight of the mother, weight of the newborn, age of the mother, type of birth, number of newborns, ethnic origin, diet food preferences and/or avoidances, whether the mother is a vegan or vegetarian, blood sugar level, the mother's temperature, and the day of week and month of the year on which the breastfeeding mother enters the data, or any combination thereof.

In certain embodiments, the cloud server 204 receives input data from a user through the user device 202, from which the computer-based breastmilk qualification system 200 may create a profile of the user. The input data may include information data, such as but not limited to, a scanned picture of the test strip 100 and additional data such as: weight of the mother; weight of the newborn; age of the mother; type of birth; number of newborns; ethnic origin; diet; the breastfeeding mother's food preferences and/or avoidances, whether she is a vegan or vegetarian, her blood sugar level, her temperature and the like.

The system 200 of the invention can be used for any desired measurement, and the computer-based algorithms can be programmed to provide any desired output, e.g. whether the breastmilk is spoiled or not, which components are missing or overloaded, etc., and also which dietary modification should be carried out in order to improve the breastmilk quality.

Accordingly, in a specific embodiment, the system 200 according to the invention uses a Breastmilk Qualification Model I 1300 to provide an output stating whether the breastmilk is spoiled or not and/or whether there any nutrients' deficiencies. In an alternative specific embodiment, the system 200 according to the invention uses a Breastmilk Qualification Model II 1400 is trained to provide output recommendations for nutritional components, quantities and consumption frequency for overcoming said nutrients deficiencies and subsequently for improving breastmilk's quality.

The present invention further provides a method for testing breastmilk to determine whether it: (i) is spoiled or not, and/or (ii) has deficiencies in certain nutrients, and for producing recommendations for nutritional components, quantities and consumption frequency for overcoming said deficiencies and improving breastmilk's quality, the method comprises the following steps: (1) providing a test strip 100 according to the invention; (2) providing a user device 202 comprising a scanning element for scanning said testing regions 112 of said test strip 100; (3) testing said breastmilk using said at least one test strips 100 and scanning said testing regions 112 using said user device 202; (4) applying machine learning algorithms at an online remote cloud server 204, said algorithms are designed to incorporate uploaded user's data and data obtained from said user device 202 and provide an output stating whether the breastmilk is spoiled or not and/or has deficiencies in certain nutrients, and further provide an output with recommendations for nutritional regimen to improve breastmilk's quality; and (5) providing an online interface to access said outputs. In specific embodiments, the method further comprises a step of providing a color chart/a reference color card that accompanies the test strip 100 and subsequently a step of comparing the color obtained in the test strip 100 to the color chart. This color chart may be an independent card or stick, or may be an integral part of the test strip 100, e.g., located on its handle or at an area near the test region(s).

Step (3) of testing the breastmilk using a test strip 100 according to the invention can be carried out at any time point, such as immediately after pumping and before freezing, after thawing frozen pumped breastmilk, during breastfeeding, etc. It should be noted that subsequent steps (4) and (5) can be carried out immediately after step (3) or at any time point after step (3), e.g. if step (3) is carried out during breastfeeding, steps (4) and (5) can be carried out after finishing the breastfeeding and/or after the baby is a sleep.

In certain embodiments of the method of the invention, the test strip 100 is designed to determine at least one of the following: breastmilk expiration, colic symptom reduction, vitamin C concentration, macronutrients concentration, baby sleep support, contamination detection, improvement in learning and memory, and antioxidant detection, or any combination thereof. In a specific embodiment, the test strip 100 is designed to determine all of the above.

As explained above, the test strip 100 of the invention comprises test regions 112 with pads for measuring and identifying various specific components and parameters in order to enable the computerized server 204 to provide an output regarding the breastmilk (e.g. spoiled or not or that certain nutritional modifications are needed). For instance, in specific embodiments of the method of the invention, the test strip 100 is used to determine: (a) breastmilk expiration and comprises a pad for measuring galactose concentration and a pad for measuring pH; (b) colic symptom reduction and comprises a pad for measuring triglyceride concentration and optionally in combination with a pad for measuring lactose concentration; (c) vitamin C concentration and comprises a pad for measuring vitamin C concentration (d) macronutrients concentration and comprises a pad for measuring the concentration of lactose, a pad for measuring the concentration of triglyceride and a pad for measuring the concentration of protein; (e) baby sleep support comprises pads for measuring the concentration of iron, magnesium, nicotine and caffeine; (f) contamination detection comprises pads for measuring the concentration of alcohol, nicotine and caffeine; (g) improvement in learning and memory comprises pads for measuring the concentration of iron, choline, magnesium and sialic acid; and/or (h) antioxidant detection comprises a pad for measuring the concentration of vitamin C and pad(s) for measuring either malondialdehyde (MDA) and/or glutathione peroxidase; or any combination thereof.

The method according to any one of the embodiments above may comprise additional steps, such as: (1) accumulating and monitoring data from at least one user device 202; and (2) propagating said data to an online remote server 204.

### EXAMPLES

### Product 1—Breastmilk Expiration Determination Strip

Breastmilk is usually suitable for consumption after storage according to storage guidelines. However, in some cases the breastmilk is considered not safe or unsuitable for consumption. Such cases are usually involved with an elevation in aero-bacterial load.

Diagnosis of such elevation in bacterial activity and bacterial load (using a direct count, CFU/ml) requires special equipment (e.g. microscope) and skill, and thus cannot be diagnosed at home. However, elevations in galactose concentration and/or D-lactic acid concentration, which indicate elevation in bacterial activity and bacterial load may be detected and diagnosed at home using simple detection means.

The galactose concentration in stored breastmilk may elevate due to the fermentation of human breastmilk oligosaccharide components by lactic acid bacteria. When the lactic acid bacteria transform the lactose in the breastmilk to glucose and galactose, it uses glucose as an energy supply molecule, and thus, some of the free galactose is accumulated.

In accordance with some embodiments of the present invention, the breastmilk expiration test strip 100 is designed to detect an elevation in bacterial load in stored breastmilk (refrigerated or thawed). The test strip may contain (a) a basic pH test pad, and (b) a complementary test pad of a bacterial load marker such as, e.g., a marker indicating the accumulation of galactose. Other bacterial load strips that may be used for this purpose are lactic acid accumulation strip (Dornic acidity units), and/or a strip indicating any reduction in protein content.

Since Fresh breastmilk does not contain galactose, the galactose concentration that indicates a high bacterial overload in breastmilk is determined and calibrated in accordance with some embodiments of the present invention.

### Sensor 1: Galactose Test Strip for Breastmilk—Preparation of a Galactose Test Strip for Breastmilk

Preparation Steps:
1) preparing solution (a) by dissolving galactose oxidase (12 U) and horseradish peroxidase (50 U) in 0.1M phosphate buffer [pH 7.0];
2) preparing solution (B) by dissolving O-dianisidine (10 mg/ml), ABTS (50 mg/ml) and cresol green (1 mg/ml) in 0.1M phosphate buffer [pH 7.0];
3) (optional) adding EDTA (1-5 mg/ml) and/or BSA (10 mg/ml) and/or triton (0.1%) and/or glutaraldehyde (0.25%) to reaction mix;
4) mixing solutions (a) and (b) and apply on a blotting paper; and
5) drying the blotting paper for 15 minutes in hot air.

In accordance with some embodiments of the present invention, the concentration of galactose in breastmilk may be tested via chemical methods by using its reductive sugar properties or via enzymatic methods by using galactose dehydrogenase and diaphorase, BSA, Triton, EDTA and/or glutaraldehyde in different concentrations and/or combinations.

In accordance with some embodiments of the present invention, testing a breastmilk sample comprises the following steps:
(a) dipping the test strip 100 in a breastmilk sample or dripping breastmilk onto the sample receiving reservoir 108 or directly onto the testing regions 120 of the test strip 100 (as described below);
(b) scanning the test strip 100 via a user device 202, such as a cellphone, accompanied with suitable application(s) for monitoring the colors of the test region(s)/pad(s); and
(c) analyzing the colors of the test region(s)/pad(s) via machine learning algorithms (as explained below), either on the user device 202, or on a remote server 204.

In the case of identifying extremely high/low pH levels, the breastmilk is considered suitable for use. However, if near normal pH values are identified, the breastmilk qualification program 206 will further monitor galactose concentrations: When the galactose concentration is relatively low, breastmilk is considered good for consumption. However, when the galactose concentration is relatively high (suggesting that the pH level is low due to upload in bacterial growth), the breastmilk is considered unsafe for use.

### Sensor 2: Lactic Acid and Dornic Acidity Detection Test Strip

Measured lactic acid amounts can be expressed in “Dornic acidity units” which are defined as the amount of lactic acid in the measured medium (1 Dornic degree (1° D) equals to 0.1 g/L lactic acid). The amount of lactic acid or “Dornic acidity” is a marker of bacterial activity that can be used as a marker for breastmilk expiration or breastmilk correct storage and pumping hygiene. In breastmilk banks, Dornic degrees are used as a marker for breastmilk quality and acceptability and safety for use.

Bacterial growth and activity in stored breastmilk results in elevation of lactic acid (D more than L) secretion. The lactate produced in human cells is almost exclusively L-lactate, and it is usually not found in breastmilk except immediately after sport activities. Bacteria, on the other hand, produce both D-lactate and L-lactate. Therefore, identification of the presence of D-lactate either alone or with L-lactate in breastmilk, may be a measure of bacterial activity and growth therein.

In accordance with some embodiments of the present invention, the lactic acid and Dornic acidity detection test strip is designed to detect and measure lactic acid (D/L) in breastmilk or to estimate bacterial activity and growth. Such measurements/estimations are essential for testing whether stored breastmilk is safe for consumption.

Protocol 2000 (FIG. 4)—Preparation of a Lactic Acid Test Strip for Breastmilk

Preparation Steps:
1) preparing a solution by mixing:
   (a) 50 mM HCl buffer [pH 8.7]
   (b) either D-lactic acid dehydrogenase (50 U/ml) or a mixture of D-lactic acid and L-lactic dehydrogenses (30 U/ml each) and
   (c) Diaphorase (5 U/ml) with NAD 2 mM,
   (d) MgCl₂ 10 mM,
   (e) Ammonium sulfate 15 mM,
   (f) Resazulin (100 μg/ml),
   (g) PEG (1 mg/ml),
   (h) BSA (10 mg/ml) and
   (i) TritonX100 (1 μl/ml).
2) applying the solution on a test strip and drying it in hot air for approximately 15 minutes; and
3) prior to using the resulting test strip with breastmilk, adding a second blotting paper dipped in glycine-hydrazine buffer [pH 9.0], and allow the breastmilk to flow through it before reaching the lactic acid test pad.

In accordance with some embodiments of the present invention, the use of the lactic acid and Dornic acidity detection test strip 100 as prepared above is accompanied with a breastmilk qualification program 206 that performs an online analysis of the test strip 100 and provides the mother with results regarding the tested breastmilk and with nutritional and practical recommendations.

### Product 2—Colic Symptom Reduction Detection Test Strip

Colic symptoms in babies highly resemble lactose overload and lactose intolerance. It is known that low maternal fat intake and high maternal sugar intake can increase both colic and lactose intolerance symptoms in breastfed babies, and it seems that the ratio between fat and lactose affects the intensity of colic and lactose intolerance symptoms.

It is also known that a low-FODMAP (Fermentable Oligo-, Di-, Mono-Saccharides And Polyols) maternal diet is associated with enhanced reduction in crying-fussing durations of infants with colic, without changing the lactose content of their breastmilk. However, in general, in a low-FODMAP diet, food with high-FODMAP is usually replaced with food that is rich in fat and as a result, a low-FODMAP diet increases the fat that the mother consumes. Hence, the ratio of lactose and fat in breastmilk may provide an insight regarding dietary recommendations that could decrease colic symptoms.

Accordingly, in accordance with some embodiments of the present invention, a colic symptoms reduction test strip 100 is designed to detect the correlation between the concentrations of different breastmilk components. The test strip 100 of the invention may be used for detecting and measuring the concentration of fat and sugars in the breastmilk, together with a breastmilk qualification program 206 that provides an analysis of the ratio between fat and sugars and its effects on colic symptoms. In addition, the program 206 may further provide the mother with tailored nutritional recommendations for optimizing the ratio of macronutrients in her breastmilk to reduce the baby's colic symptoms.

### Sensor 3: Lactose Test Strip for Breastmilk

Preparation of Lactose Test Strip for Breastmilk:

1) preparing solution (a) by dissolving b-galactosidase (20 U), glucose oxidase (20 U) and horseradish peroxidase (200 U) in 0.1M citrate buffer [pH 6.2]; enzyme concentrations were chosen to fit the proper range of lactose in breastmilk samples
2) preparing solution (b) by dissolving ABTS (2 mg/ml) and cresol green (1 mg/ml) in 0.1M citrate buffer [pH 6.2];
3) mixing solutions (a) and (b) and apply on blotting paper;
4) drying the blotting paper in hot air chamber in atmospheric pressure for 15 minutes;
5) optimizing the combination of pads colorimetric reaction to breastmilk natural color;
6) applying a solution of O-dianisidine dissolved in 0.1M citrate buffer [pH 6.2] (2.5 mg/ml) to the dried blotting paper and drying the paper once again in hot air chamber in atmospheric pressure for 15 minutes.

Lactose concentration in breastmilk may be determined using various techniques. For instance, different enzymatic reactions, such as lactose dehydrogenase and diaphorase can be used. Alternatively, or additionally, chemical reactions can be used, such as Fehling's test, estimation of lactose utilizing trichloroacetic acid (TCA) as a total protein precipitating agent, lactose reacted with methylamine in hot alkaline solution, and phenylhydrazine hydrochloride/sodium metabisulfite/acetic acid.

In certain embodiments, the breastmilk may be tested with or without dilution, e.g., in water to best fit the visible detection range of the test strips.

In further embodiments, BSA-, Triton-, EDTA- or glutaraldehyde-pads, in various combinations and concentrations may be added to the test strip 100 of the invention for standardized comparison. In alternative embodiments, the test strip 100 of the invention are accompanied with standardization test strips comprising BSA, Triton, EDTA or glutaraldehyde in various combinations and concentrations, for breastmilk nutritional composition analysis.

### Sensor 4: Triglyceride Test Strip for Breastmilk

The following protocols may be used for preparing triglycerides test strips for breastmilk that are highly suitable with the complex nutritional composition of breastmilk:

Preparation 1:
1) mixing in 50 mM HCl Buffer [pH 8.7]: lipoprotein lipase (2000 U/ml), glycerol dehydrogenase (30 U/ml) and diaphorase (2 U/ml) with NAD 2 mM, MgCl₂ 10 mM, ammonium sulfate 15 mM, resazulin (150 μg/ml), PEG (1 mg/ml), BSA (10 mg/ml) and TritonX100 (1 μl/ml);
2) applying the mixture on a blotting paper; and
3) drying in hot air chamber in atmospheric pressure for about 15 minutes.

Preparation 2:
1) preparing a solution from water (405 ml), TritonX100 (1 g), CHAPS (1 g), poly methyl vinyl ether (21 g), calcium chloride (0.2 g), Na-ATP (32 g), sucrose (25 g) and HPC (hydroxypropyl cellulose) foundation* and adjusting the pH to 5.7;
2) mixing in the prepared solution: lipoprotein lipase (625 U), G3P oxidase (glycerol-3-phosphate oxidase) (500 U), glycerol kinase (359 U) and horseradish peroxidase (100 U), with TMBZ (6.25 g) and 4-AAP (20 g) and EDTA (5 g);
3) applying the mixture on a blotting paper; and
4) drying in hot air chamber in atmospheric pressure for about 15 minutes.

Preparation 3:
1) preparing a solution of 1.5 mg/ml Oil-Red-o and/or Sudan-Black, Indigo Blue, in water, and homogenizing for 30 seconds using electric homogenizer to create an homogenous emulsion;
2) applying the solution to a filter paper; and
3) drying in hot air chamber in atmospheric pressure for about 15 min.

The resulting paper can then be cut into equal-sized strip pads that can be used for testing breastmilk. The testing can be carried out by dipping the pads in breastmilk and let the breastmilk be absorbed and reach all parts of the pad. After 5-10 min the area and/or color intensity, can be measured and calculated.

In certain embodiments, in order to allow a sensitive visible detection range of triglycerides in breastmilk, different food dyes may be added. For example: 1% sunset yellow FCF or 0.5% fast green FCF.

In accordance with some embodiments of the present invention, the use of the triglyceride test strip 100 as prepared above is accompanied with a breastmilk qualification program 206 that performs an online analysis of the test strip 100 and provides the mother with information regarding her breastmilk and with nutritional recommendations for enhancing breastmilk's production and for decreasing colic symptoms.

### Product 3—Vitamin C Concentration Determination Test Strip

Vitamin C is an antioxidant vitamin that has a number of biochemical functions linked to the function of the immune system. It is considered as a natural barrier against infection, stimulates leukocytes for the phagocytic and anti-microbial activity, augments antibody production and complement levels. Vitamin C also helps in iron absorbance and enhances synthesis of interferons that fights viral infections. Hence, vitamin C is vital for baby growth and development, immune system development and function, and general development and survival.

The concentration of vitamin C decreases on average by one-third within 24 hours in breastmilk stored in the refrigerator and by one or two thirds in breastmilk stored in the freezer. Such high decreasing rates may lead to a complete loss of vitamin C in stored breastmilk.

It has been found that the normal concentration of vitamin C in breastmilk ranges from 2.8 to 6 mg/dL, and that its concentration in the breastmilk increases 30 min. after the consumption of vitamin C by the mother.

Vitamin C may be detected by via a chemical reaction, and in accordance with some embodiments of the present invention, the vitamin C concentration strip 100 is designed to detect and measure the concentration of vitamin C in breastmilk.

### Sensor 5: Vitamin C Test Strip for Breastmilk

Preparation:
1) preparing solution (a) by mixing EDTA-2Na (5 g), ferric chloride (2 g), sodium citrate (10 g), and food dye blue No. 1 (0.3 ml) with DDW+NaOH [pH10], and bringing the volume to 10 ml;
2) preparing solution (b) by mixing 2,2″-bipyridine (0.2 g), PVP (0.25 g) and 20 ml EtOH;
3) applying solution (a) to a blotting paper and drying it in hot air chamber in atmospheric pressure for 15 min.;
4) applying solution (b) to the blotting paper of step (3) and drying in hot air chamber in atmospheric pressure for 15 minutes.

In accordance with some embodiments of the present invention, the concentration vitamin C in breastmilk may be tested via addition of different food coloring to the reaction or 0.1 mg/ml cresol green to solution (b) to change the reaction color.

In accordance with some embodiments of the present invention, the use of the vitamin C test strip 100 is accompanied with a breastmilk qualification program 206 that performs an online analysis of the strip 100 and provides the mother with results regarding the tested breastmilk and with nutritional and practical recommendations.

### Product 4—Macronutrients Concentration Detection Test Strip

In accordance with some embodiments of the present invention, the concentrations of nutrients in breastmilk such as lactose, triglycerides and protein are determined using the breastmilk macronutrients concentration test strip 100.

Breastmilk comprises about: 1% protein, 6.9% carbohydrate and 4.4% fat by weight, or 5.6% protein, 38.8% carbohydrate and 55.6% fat by calories. In accordance with some embodiments of the present invention, the breastmilk macronutrients concentration test strip 100 is designed to detect and measure the concentration of the three main nutrients in breastmilk, i.e., (a) lactose, (b) triglycerides and (c) protein. The appropriate concentration of these macronutrients is essential for the baby growth and development.

Accordingly, in certain embodiments, the strip 100 of the invention contains three detection pads: (a) a lactose pad as described above in Product 2; (b) a triglyceride pad as described above in Product 2; and (c) a protein detection and measuring pad.

### Product 5—Baby Sleep Support Test Strip

Breastmilk contains many components and substances that are associated with sleep habits such as iron, magnesium, caffeine and nicotine. As such, in certain embodiments, the present invention provides test strips for measuring the content and amount of such components and substances in order to provide recommendations for parents with a baby that suffers from sleeping problems.

### Sensor 7: Magnesium Detection Pad

Magnesium can be detected, for example, by using a Xylidyl Blue Magnesium Assay or by using Calmagite in alkaline solution. Accordingly, in accordance with some embodiments of the test strip 100 of the present invention, the Xylidyl Blue Magnesium Assay is optimized for measuring the concentration of magnesium in breastmilk. Alternatively, Calmagite measurement is optimized for measuring the concentration of magnesium in breastmilk.

### Sensor 8: Iron Detection Pad

Iron can be detected via known colorimetric methods calibrated for iron concentrations in breastmilk. The common chlorophores used are: TPTZ (2,4,6-Tris(2-pyridyl)-s-triazine); Ferrozine; 1,10-phenanthroline/2,2′-bipyridine; Fluorinated-β-Diketone; Disodium-1,2-dihydroxybenzene-3,5-disulfonate; and Thio-β-diketones. Accordingly, in accordance with some embodiments of the test strip 100 of the present invention, the above mentioned chlorophores are incorporated in a pad(s) for measuring iron concentration in breastmilk.

### Sensor 9: Nicotine Detection Pad

Nicotine can be detected by immunoassay, e.g. using an antibody for nicotine in a lateral ELISA assay, or by an enzymatic assay. Enzymatic assays include the use of nicotine-dehydrogenase; (S)-6-hydroxynicotine oxidase or (R)-6-hydroxynicotine oxidase, and HRP reaction. Accordingly, in certain embodiments, the test strip 100 of the present invention comprises pad(s) for detection of nicotine content and concentration based on either immune- or enzymatic-assay.

### Sensor 10: Caffeine Detection Pad

Caffeine can be detected by immunoassay, e.g. using an antibody for caffeine in a lateral ELISA assay, or by an enzymatic assay. Accordingly, in certain embodiments of the test strip 100 of the invention, a caffeine detection pad comprises an antibody for caffeine for a lateral ELISA assay for determination of average caffeine levels in breastmilk, or comprises an enzymatic reaction that includes any one of: a caffeine dehydrogenase or oxidase, dichlorophenol, indophenol, coenzyme Q₀, and cytochrome C and 50 mM potassium phosphate buffer.

### Sensor 11: Melatonin Detection Pad

Detection of melatonin in breast milk will be performed by one or more of the following methods, being adjusted and optimized for breast milk complexity and concentrations: (i) lateral ELIZA; or (ii) determination of melatonin concentration by utilizing the reactions of p-dimethylaminobenzaldehyde in hydrochloric acid (van Urk reagent)-ferric chloride in sulphuric acid (Salkowski reagent) mixture. The blue color of the resulting reaction product is measured at 630 nm.

The combination of sensors 7-11 above, either on multiple test strips or on a single test strip provides data that can be used for evaluating baby sleep quality and provide support on ways to improve it.

It should be noted that a test strip 100 according to the invention for baby sleep support can comprise either all of sensors 7-11 or part thereof.

### Product 6—Contamination Test Strip

A contamination test strip according to the present invention is designed to detect and measure various contaminants in breastmilk, such as high levels of alcohol, presence of nicotine, and level of caffeine. For doing so, the strip 100 contains 1, 2, 3 or more detection pads, each designed to measure one of the desired contaminants. For instance, a nicotine pad as described above under Sensor 9; a caffeine pad as described above under Sensor 10, and an alcohol test pad as described below under Sensor 12.

### Sensor 12: Alcohol Test Strip for Breastmilk

Preparation:
1) preparing an enzyme mixture by mixing alcohol oxidase, HRP and 20 mM Tris-HCl (0.2% BSA) [pH 7.5]; and
2) preparing a chromagen mixture by mixing: O-dianisidine, ABTS and 20 mM Tris-HCl (0.2% BSA) [pH 7.5].

### Product 7—A Test Strip for Testing Improvements in Learning and Memory in the Infant

An improvement in learning and memory test strip according to the present invention is designed to detect and measure various parameters and components within breastmilk, such as magnesium levels, Iron levels, choline levels and sialic acid presence and level. For doing so, the strip 100 contains 1, 2, 3 or more detection pads, each designed to measure one of the desired parameters and components. For instance, a magnesium pad as described above under the protocol of Sensor 7, an iron pad as described above under the protocol of Sensor 8, and a sialic acid pad.

### Product 8—Antioxidant Detection Test Strip

For detecting and measuring antioxidants in breastmilk, a test strip 100 according to certain embodiments of the invention comprises a combination of test regions with pads for measuring parameters and components associated with antioxidants, such as a pad for measuring vitamin C together with a pad for measuring/detecting malondialdehyde (MDA) or a pad for measuring/detecting glutathione peroxidase.

### Training Protocols

FIG. 4 is a block diagram depicting a high level overview of a system 200 according to some embodiments of the invention, wherein a plurality of training set modules 1200 extract information pertaining to the pH of the breastmilk, the concentrations of the chemical species of the breastmilk as well as other parameters and propagate this data to a main server 204 according to some embodiments of the present invention.

A training set module 1200 is a module implemented in software or hardware or any combination thereof, installed at the user device 202 and/or the server 204. The module 1200 is configured to interface the server module 204 using any type of wired or wireless data communication standard (e.g. LAN, WAN, WiFi, GSM, 3GPP, LTE, etc.), and convey to the server 204 data pertaining to the user.

In certain embodiments, and as illustrated in FIG. 4, the module 1200 is comprised of at least one of the following sub modules: pH module 2100; bacteria load such as galactose module 2200; lactose module 2300; triglyceride module 2400; vitamin C module 2500; lactic acid and Dornic acidity module 2600; protein module 2700; other vitamins, minerals and additional chemicals module 2800; Other parameters module 2900; and the day of week and month of the year module 3000.

In certain embodiments, the module 204 includes several sub modules, configured to accumulate data, analyze the data, and inform the user of when and what to consume in order to overcome nutritional deficiencies and thus to improve the breastmilk quality. Non-limiting examples of such sub-modules include: data acquisition module 1100; training module 1200; Model I 1300; Model II 1400; and decision module 1500, or any combination thereof.

In specific embodiments, the data acquisition module 1100 accumulates data from the training set modules 1200, and stores it in a database for further processing, said data includes at least one of the following measurements: pH; Concentration of bacteria load such as galactose; Concentration of lactose; Concentrations of triglyceride; Concentrations of vitamin C; Concentrations of lactic acid and Dornic acidity; Concentrations of protein; Concentrations of other vitamins, minerals and additional chemicals such as zinc, magnesium, iron, vitamin B12, and nitrogen; Other parameters; and the day of week and month of the year, or any combination thereof.

### Data Acquisition Module 1100

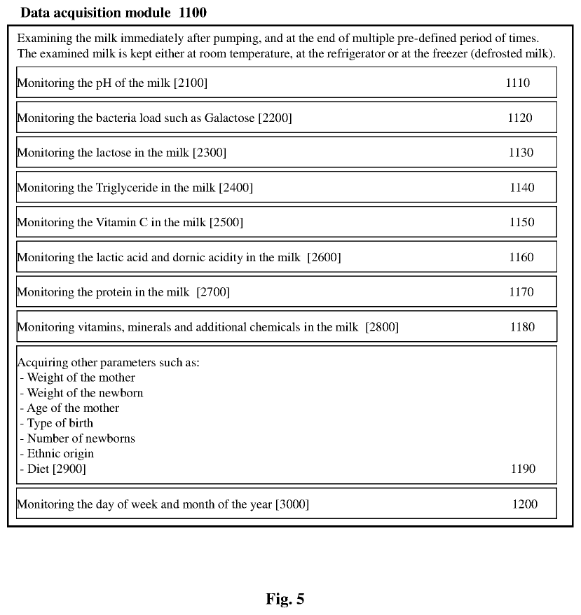

FIG. 5 is a flow diagram depicting the function of a data acquisition module 1100 according to some embodiments of the invention. This module 1100 resides within the server 204 and accumulates data relevant to the breastfeeding mother and to the breastmilk. The breastmilk is examined immediately after pumping, or at the end of multiple pre-defined period of times. The examined breastmilk may be kept either at room temperature, in the refrigerator or in the freezer (defrosted breastmilk).

The data acquisition module 1100 aggregates and stores at least part of the following data in a database for further analysis (or any combination thereof): (1) data of pH measurements of the breastmilk is acquired at pre-determined time periods from the pH module 2100 (step 1110); (2) data of bacteria load such as galactose in the breastmilk is acquired at pre-determined time periods from the bacteria load module 2200 (step 1120); (3) data of the lactose in the breastmilk is acquired at pre-determined time periods from the lactose 2300 (step 1130); (4) data of the triglyceride in the breastmilk is acquired at pre-determined time periods from the triglyceride module 2400 (step 1140); (5) data of vitamin C in the breastmilk is acquired at pre-determined time periods from the vitamin C module 2500 (step 1150); (6) data of lactic acid and dornic acidity in the breastmilk is acquired at pre-determined time periods from the lactic acid and dornic acidity module 2600 (step 1160); (7) data of protein in the breastmilk is acquired at pre-determined time periods from the protein module 2700 (step 1170); (8) data of the vitamins, minerals and additional chemicals such as, but not limited to, sodium, zinc, iron, vitamin B12 iodine, nicotine, caffeine, alcohol, cannabis, thiamin, vitamin D, vitamin B1, in the breastmilk is acquired at pre-determined time periods from the vitamins, minerals and additional chemicals module 2800 (step 1180); (9) data of the following additional parameters is acquired at pre-determined time periods from the other parameters module 2900 (step 1190): mother's weight, newborn's weight; mother's age; type of birth; number of newborns; ethnic origin; and diet, or any combination thereof; and (10) the day of week and month of the year on which the breastfeeding mother enters the data is acquired at the day of week and month of the year module 3000 (step 1200).

### Training Modules 1200

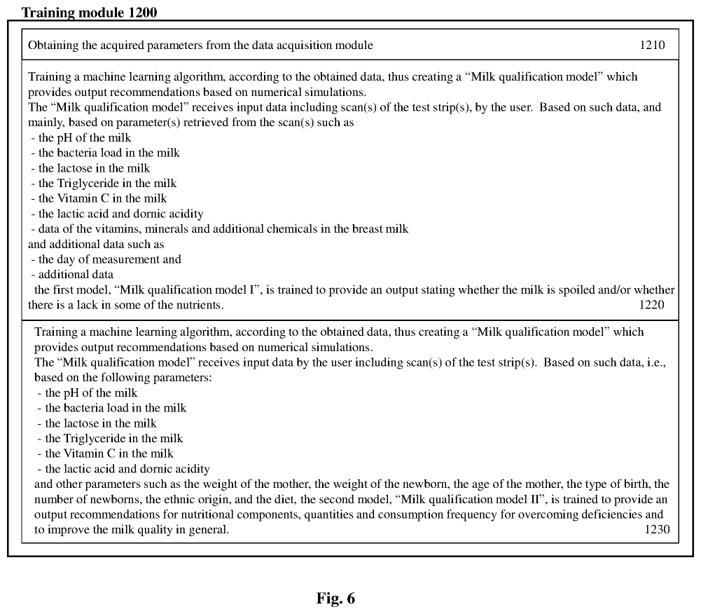

FIG. 6 is a flow diagram depicting the functionality of a training module 1200 according to some embodiments of the present invention. In certain embodiments, this training module 1200 resides within the server 204 and is responsible for training or calibrating a machine learning and/or rule based algorithm in relation to the breastfeeding mother in the training set module 1200 based on the following obtained data: the bacteria load in the breastmilk; the lactose in the breastmilk; the Triglyceride in the breastmilk; the Vitamin C in the breastmilk; the lactic acid and Dornic acidity; data of the vitamins, minerals and additional chemicals in the breastmilk; the day of measurement; additional data such as but not limited to weight of the mother, weight of the newborn, age of the mother, type of birth, number of newborns, ethnic origin, diet and the like, or any combination thereof.

Based on the above obtained data, the module 1200 creates a first model, referred to herein as “Milk Qualification Model I” 1300, which provides output recommendations based on numerical simulations. This Milk Qualification Model I 1300 receives input data by the user, and based thereon is trained to provide an output stating whether the breastmilk is spoiled or not and/or whether there is a lack in some of the nutrients. In specific embodiments, Model I 1300 is also based on the following parameters, or any combination thereof: the pH of the breastmilk; the bacteria load in the breastmilk; the lactose in the breastmilk; the triglyceride in the breastmilk; the vitamin C in the breastmilk; the lactic acid and Dornic acidity; and other parameters such as the weight of the mother, the weight of the newborn, the age of the mother, the type of birth, the number of newborns, the ethnic origin, and the diet.

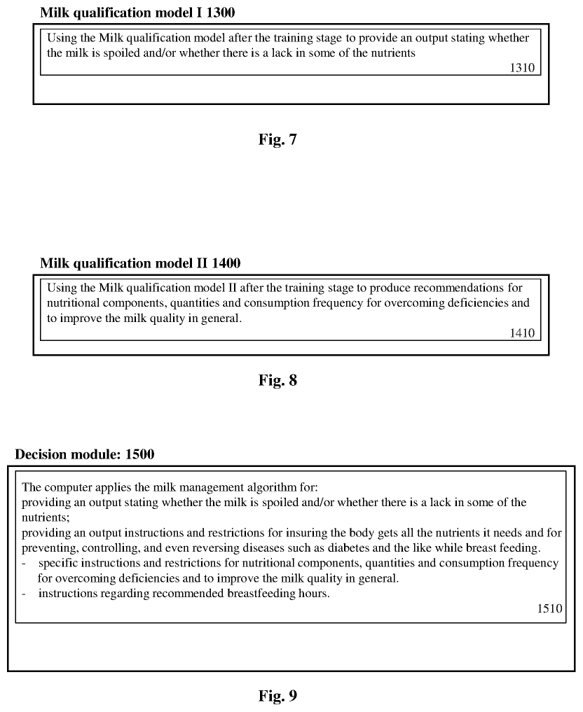

FIG. 7 is a flow diagram depicting the function of Model I 1300 after the training stage (step 1310). The Model 1300 is used after the training stage to produce an output stating whether the breastmilk is spoiled or not and/or whether there is a lack in some of the nutrients.

A second model, referred to herein as “Milk Qualification Model II” 1400 is trained to provide output recommendations for nutritional components, quantities and consumption frequency for overcoming nutritional deficiencies, and thus, to improve the breastmilk quality in general.

FIG. 8 is a flow diagram depicting the function of Model II 1400 after the training stage (step 1410). The Model 1400 is used after the training stage to produce recommendations for nutritional components, quantities and consumption frequency for overcoming deficiencies and to improve the breastmilk quality in general.

Based on numerical simulations, Breastmilk Qualification Model I 1300 and Breastmilk Qualification Model II 1400 provide output recommendations (steps 1220 & 1230) stating whether or not the breastmilk is spoiled or not, and further provide output recommendations for nutritional components, quantities and consumption frequency for overcoming nutrition deficiencies and improving the breastmilk quality in general.

FIG. 9 is a flow diagram, depicting the functionality of a decision module 1500 according to some embodiments of the present invention. This module 1500 resides within server 204 and applies an algorithm for: (1) providing an output stating whether or not the breastmilk is spoiled and/or whether there is a lack in some of the nutrients, and (2) producing recommendations for nutritional components, quantities and consumption frequency for overcoming nutritional deficiencies and to improve the breastmilk quality in general.

It should be noted that the breastmilk qualification system 200 according to the invention may provide results of tests conducted during a pre-defined period of time, for instance, during a period of 6 months, in the form of graphs, tables and any other presentation format for enabling the feeding mother to examine the effect of her eating habits on her breastmilk.

In addition, breastmilk qualification system 200 may provide instant feedback and may enable users other than the mother to get some of the information, e.g., some of the data may be accessible to users (health care providers and the like) other than the mother. Additionally, the breastmilk qualification system 200 of the invention may transfer the results to the attending physician and may provide statistics, reports and graphic illustrations of the data.

In addition, it may be possible to compare the results obtained via breastmilk qualification system 200 to blood test results for generating additional insights and improving the diet of the breastfeeding mother.

1. A test strip 100 for testing a breastmilk sample, the test strip 100 comprising a working surface 104 that comprises at least one testing region 112 with at least one testing medium, each one of said at least one testing medium is designed for measuring one component or parameter of the breastmilk, said at least one testing medium comprises: (i) at least one testing pad for enabling a single-step measuring procedure; and/or (ii) at least two testing pads for enabling a multi-step measuring procedure,
wherein said testing regions 112 are designed to undergo color change and/or texture/granulation variations as a result of a colorimetric reaction inducible when exposed to said breastmilk.

2. The test strip 100 according to claim 1, wherein said color change means a change in color intensity, hue, tone, shade, or any combination thereof.

3. The test strip 100 according to claim 1, wherein said test strip 100 comprises at least one of:
breastmilk expiration testing region;
colic symptom reduction testing region;
vitamin C concentration testing region;
macronutrients concentration testing region;
baby sleep support testing region;
contamination detection testing region;
improvement in learning and memory testing region; and
antioxidant detection testing region,
or any combination thereof.

4. The test strip 100 according to claim 3, wherein said breastmilk expiration testing region comprises a pH test pad, and/or a bacterial load marker pad.

5. The test strip 100 according to claim 4, wherein said bacterial load marker pad is selected from: a galactose test pad, a lactic acid test pad, and a protein test pad, or any combination thereof.

6. The test strip 100 according to claim 3, wherein said colic symptom reduction testing region comprises a triglyceride test pad and optionally a lactose test pad.

7. The test strip 100 according to claim 3, wherein said vitamin C concentration testing region comprises one vitamin C test pad or two different-color vitamin C test pads.

8. The test strip 100 according to claim 3, wherein said macronutrients concentration testing region comprises a lactose test pad, a triglycerides test pad, and a protein test pad.

9. The test strip 100 according to claim 3, wherein said baby sleep support testing region comprises at least one of: an iron pad, a magnesium pad, a caffeine pad and a nicotine pad.

10. The test strip 100 according to claim 3 wherein said contamination detection testing region comprises at least one of: a caffeine pad, a nicotine pad and an alcohol pad.

11. The test strip 100 according to claim 3, wherein said improvement in learning and memory testing region comprises at least one of: an iron pad, a choline pad, a magnesium pad and a sialic acid pad.

12. The test strip 100 according to claim 3, wherein said antioxidant detection testing region comprises a vitamin C pad and at least one of: malondialdehyde (MDA) pad and glutathione peroxidase pad.

13. A test strip 100 according to claim 1 for testing a breastmilk sample, wherein the working surface 104 comprises:
a sample receiving reservoir 108;
testing regions 112 comprising at least one testing medium, each one of said at least one testing medium comprises: (i) at least one testing pad for enabling a single-step measuring procedure; and/or (ii) at least two testing pads for enabling a multi-step measuring procedure; and
fluid continuous transporting routs 110;
wherein said fluid continuous transporting routs 110 connect said sample receiving reservoir 108 to each one of said testing regions 112 and are designed for directing breastmilk from said breastmilk sample placed within said reservoir 108 to said testing regions 112.

14. A system 200 for testing breastmilk to determine whether it: (i) is spoiled or not, and/or (ii) has deficiencies in certain nutrients, and for producing recommendations for nutritional components, quantities and consumption frequency for overcoming said deficiencies and improving breastmilk's quality, the system 200 comprises:
the test strip 100 according to claim 1;
optionally, a color chart/a reference color card;
a user device 202 comprising a scanning element for scanning said testing regions 112 of said test strip 100; and
a cloud server 204 including a breastmilk qualification program 206, said cloud server 204 designed to: (i) receive user's data and scan(s) of said testing regions 112 via said user device 202; (ii) apply machine learning algorithms to incorporate said data and scan(s); and (iii) provide the following outputs: whether the breastmilk is spoiled or not, and/or possible nutrients deficiencies, and recommendations for nutritional components, quantities and consumption frequency for overcoming said nutrients deficiencies for improving breastmilk's quality,
wherein said scan(s) showing color change intensities induced by colorimetric reaction(s) in said testing medium of said test strip 100, and
wherein said machine learning algorithms monitor the color change in said testing regions 112, and based thereon provide said output.

15. The system 200 according to claim 14, wherein said user's data comprises at least one of:
weight of the mother,
weight of the newborn,
age of the mother,
type of birth,
number of newborns,
ethnic origin,
diet food preferences and/or avoidances,
whether the mother is a vegan or vegetarian,
blood sugar level,
the mother's temperature, and
the day of week and month of the year on which the breastfeeding mother enters the data,
or any combination thereof.

16. The system 200 according to claim 14, wherein a Breastmilk Qualification Model I 1300 is trained to provide an output stating whether the breastmilk is spoiled or not and/or whether there any nutrients' deficiencies and/or wherein a Breastmilk Qualification Model II 1400 is trained to provide output recommendations for nutritional components, quantities and consumption frequency for overcoming said nutrients deficiencies and subsequently for improving breastmilk's quality.

17. A method for testing breastmilk to determine whether it: (i) is spoiled or not, and/or (ii) has deficiencies in certain nutrients, and for producing recommendations for nutritional components, quantities and consumption frequency for overcoming said deficiencies and improving breastmilk's quality, the method comprises
providing a test strip 100 according to claim 1;
optionally, providing a color chart/a reference color card;
providing a user device 202 comprising a scanning element for scanning said testing regions 112 of said test strip 100;
testing said breastmilk using said at least one test strips 100 and scanning said testing regions 112 using said user device 202;
applying machine learning algorithms at an online remote cloud server 204, said algorithms are designed to incorporate uploaded user's data and data obtained from said user device 202 and provide an output stating whether the breastmilk is spoiled or not and/or has deficiencies in certain nutrients, and further provide an output with recommendations for nutritional regimen to improve breastmilk's quality; and
providing an online interface to access said outputs.

18. The method according to claim 17, wherein said test strip 100 is designed to determine at least one of the following:
breastmilk expiration,
colic symptom reduction,
vitamin C concentration,
macronutrients concentration,
baby sleep support,
contamination detection,
improvement in learning and memory, and
antioxidant detection,
or any combination thereof.

19. The method according to claim 18, wherein:
said test strip 100 for determining breastmilk expiration comprises a pad for measuring galactose concentration and a pad for measuring pH;
said test strip 100 for determining colic symptom reduction comprises a pad for measuring triglyceride concentration and optionally also a pad for measuring lactose concentration;
said test strip 100 for determining vitamin C concentration comprises one pad for measuring vitamin C concentration or two different-color vitamin C test pads;
said test strip 100 for determining macronutrients concentration comprises a pad for measuring the concentration of lactose, a pad for measuring the concentration of triglyceride and a pad for measuring the concentration of protein;
said test strip 100 for determining baby sleep support comprises pads for measuring the concentration of iron, magnesium, nicotine and caffeine;
said test strip 100 for determining contamination detection comprises pads for measuring the concentration of alcohol, nicotine and caffeine;
said test strip 100 for determining improvement in learning and memory comprises pads for measuring the concentration of iron, magnesium and sialic acid; and/or
said test strip 100 for determining antioxidant detection comprises a pad for measuring the concentration of vitamin C and pad(s) for measuring either malondialdehyde (MDA) and/or glutathione peroxidase.

20. The method according to any one of claim 19, further comprising the following steps:
accumulating and monitoring data from at least one user device 202; and
propagating said data to an online remote server 204.
